(19) United States
(12) Patent Application Publication
(10) Pub. No.: US 2009/0263514 A1
(43) Pub. Date: Oct. 22, 2009

(54) Cold and flu tonic

(76) Inventor: Govindan Gopinathan, Oradell, NJ (US)

Correspondence Address: Govindan Gopinathan, MD, 680 Briarwood Court, Oradell, NJ, 07649, US

(21) Appl. No.: 12/290,739

(22) Filed: Nov. 3, 2008

Related U.S. Application Data
(60) Provisional application No. 61/124,965, filed on Apr. 21, 2008.

(51) Int. Cl. A61K 36/67; A61P 31/00

(52) U.S. Cl. 424/734

(57) ABSTRACT

A method and composition for reducing the symptoms of the common cold and flu in a human subject in need thereof, comprising the administration of an effective amount of an aqueous composition having all components dispersed as a dry powder concentrate or dissolved in a decoction therein comprising Black pepper, Cinnamon, Cumin, Ginger, Rose hip, Saffron, and Turmeric with Maple Syrup as a sweetener.

### CROSS-REFERENCES TO RELATED APPLICATIONS

This application is based on provisional application No. 61/124,965 filed Apr. 21, 2008 and claim is made for the benefit of the filing date of the provisional application.

### STATEMENT IN TRUTH

Per FDA requirements, the invention described here-in has been developed as a dietary supplement to effect the “structure or function” of the human body and the “well-being” achieved by its consumption. The statements here-in are truthful and not misleading to the best of the inventor's knowledge. Unless or until this invention is evaluated as a product functioning under the category of a drug, the following label will be provided as an attachment to the product in sale in relationship of any claims made on this invention: “The statement of claims has not been evaluated by the Food and Drug Administration. This product is not intended to diagnose, treat, cure, or prevent any disease”. In truth, this invention has not been created to diagnose, treat, cure or prevent the common cold and/or flu. Its intention is to provide relief and comfort from the symptoms of the common cold and/or flu and to assist in the reduction of the length of time in which such symptoms are experienced.

### STATEMENT REGARDING FEDERALLY SPONSORED RESEARCH AND DEVELOPMENT

Not Applicable.

### REFERENCES CITED

| Patent No. | Date | Inventor | US Code |
|---|---|---|---|
| 4,402,950 | September 1983 | Wolf, et al. | 424/734 |
| 4,409,245 | October 1983 | Wolf, et al. | 426/9 |
| 4,592,910 | June 1986 | Wolf, et al. | 424/195.1 |
| 4,595,593 | June 1986 | Wolf, et al. | 424/195.1 |
| 5,248,503 | September 1993 | Emanuel-King | 424/737 |
| 5,494,668 | February 1996 | Patwardham | 424/195.1 |
| 5,536,506 | July 1996 | Majeed, et al. | 424/464 |
| 5,560,912 | October 1996 | Neeman, et al. | 424/195.1 |
| 5,643,623 | July 1997 | Schmitz, et al. | 426/73 |
| 5,716,928 | February 1998 | Benet, et al. | 514/11 |
| 5,744,161 | April 1998 | Majeed, et al. | 424/646 |
| 5,972,382 | October 1999 | Majeed, et al. | 424/464 |
| 6,024,960 | February 2000 | Kharazmi, et al. | 424/195.1 |
| 6,027,716 | February 2000 | Levin, et al. | 424/58 |
| 6,048,533 | April 2000 | Nguyen | 424/195.1 |
| 6,121,234 | September 2000 | Benet, et al. | 514/11 |
| 6,224,871 | May 2001 | Hastings, et al. | 424/195.1 |
| 6,264,995 | July 2001 | Newmark, et al. | 424/725 |
| 6,312,736 | November 2001 | Kelly, et al. | 424/734 |
| 6,387,416 | May 2002 | Newmark, et al. | 424/725 |
| 6,391,346 | May 2002 | Newmark, et al. | 424/756 |
| 6,399,114 | June 2002 | Foreman | 424/725 |
| 6,416,807 | July 2002 | Yamamato | 426/597 |
| 6,465,019 | October 2002 | Boc, et al. | 424/736 |
| 6,544,564 | April 2003 | Farley | 424/729 |
| 6,586,018 | July 2003 | Fasano | 424/746 |
| 6,592,896 | July 2003 | Rosenbloom | 424/464 |
| 6,596,313 | July 2003 | Rosenbloom | 424/464 |
| 6,613,362 | September 2003 | Watson, et al. | 424/732 |
| 6,641,846 | November 2003 | Zhang, et al. | 424/725 |
| 6,713,115 | March 2004 | Dong | 426/589 |
| 6,767,563 | July 2004 | Farley | 424/729 |
| 6,827,945 | December 2004 | Rosenbloom | 424/464 |
| 6,949,260 | September 2005 | Knumhar | 424/601 |
| 7,087,250 | August 2006 | Marchioni | 424/618 |
| 7,166,435 | January 2007 | Rosenbloom | 435/6 |
| 7,205,010 | April 2007 | Sha | 424/756 |
| 7,241,461 | July 2007 | Myhill, et al. | 424/729 |
| 7,371,389 | May 2008 | Keefe, et al. | 424/195.17 |
| 7,384,654 | June 2008 | Menon, et al. | 424/739 |
| 7,384,655 | June 2008 | Myhill, et al. | 424/729 |
| 7,384,656 | June 2008 | Menon, et al. | 424/739 |
| 7,396,546 | July 2008 | Rosenbloom | 424/729 |
| 7,405,046 | July 2008 | Rosenbloom | 435/6 |
| 7,414,079 | August 2008 | Surburg, et al. | 514/715 |

### BACKGROUND OF THE INVENTION

1. Field of the Invention

Common Cold and Influenza are centuries old illnesses. A variety of treatment modes were used all over the world; these were predominantly symptomatic, palliative treatment. There are no definitive treatment, as of yet, for both these diseases. Both these illnesses cause fever, dehydration, fatigue, malaise and cough. Staying hydrated and loosening up the mucus and phlegm to make it easy to cough it up is crucial in the well being of the patient. Organizations like the American Lung Association, advises against using tea, coffee, colas, and alcoholic beverages in common cold patients. The author is presenting in this patent, an effective herbal drink, which will not only replace fluids loss, but also provide the patient with nutrition (calories) and amelioration of throat congestion and inflammation along with enhancing the appetite. The “Cold and Flu Tonic” is designed to be used daily, in quantities needed to keep patient well hydrated and nourished, in addition to provide some healing effect to the illness the person is suffering from.

Common Cold, also called Acute Coryza or Acute Viral Nasopharyngitis is a viral infection of the upper respiratory pathways, caused by rhinoviruses (picarnovirus) or coronaviruses. It is highly contagious, spread by droplet infection mode (coughing and sneezing) or by direct contact of nasal secretions or saliva of an infected cold sufferer. The common Symptoms are fever, body aches, sore throat, runny nose and nasal congestion, cough and sneezing. Head ache, “pink” eyes, fatigue, malaise, and loss of appetite could be present. The whole illness usually clears up in 7 days, but not uncommonly could last up to two weeks.

The economic impact of common cold to the society could be staggering. In the U.S. there are 62 million afflicted with cold each year, but each individual could get more than one “cold attack” per year: children get 6-10 a year, adults 24 and the elderly one attack per year. Thus, the total number of “cold attacks” in the U.S. per year could be up to 1 billion Children miss 22 million school days a year because of the cold. Women get more cold attacks than men, because of their closer proximity to children. Cold is responsible for 75 to 100 million MD visits, annually, at a cost of $7.7 billion annually. Cold sufferers in the U.S alone spend $2.9 billion on over-the-counter (OTC) medications and spend nearly $400 million on prescription medications. Adult cold sufferers miss 150 million work days and another 126 million work days are lost when parents take off days to care for their children suffering from common cold. The total economic impact on common cold to the U.S, is in excess of $20 billion, annually.

There are no proven, effective treatments for common cold. Antibiotics are not proven to be useful in influencing the clinical course of cold, but are useful to treat secondary infections. Nevertheless, 41 million antibiotic prescriptions are written, annually, for common cold, at a cost of $1.1 billion. The National Institute of Allergy and Infectious Diseases recommends hydration, by drinking fluids, nasal sprays, gargles with warm saline water, and OTC pain and cold remedies. The American Lung Association advices against drinking coffee, tea, cola drinks with caffeine, and alcoholic beverages. Pain medications like Tylenol (acetaminophen) and Aspirin, mega doses of Vitamin-C, Zinc preparations, decongestants like Pseudo-ephedrine (Sudafed) anti-histamines like Benadryl (Diphenhydramine) and cough suppressants like Vicks and Codeine Sulfate are some of the commonly used medications to treat cold. Recently anti-viral drugs example: Tammiflu is introduced, but they are useful only if taken before the onset of cold symptoms. Herbal medications are extensively used in treating cold, all over the world. Echinacea, Chamomile (herbal tea) Garlic, Ginger, Lemon, and Liquorice are some of them.

Influenza, also called “Flu” is another acute infectious disease caused by viruses belonging to the family of Orthomyxoviridae (influenza Viruses), first discovered in pigs by Richard Schope in 1931, isolation of the virus from the humans was accomplished by Patrick Laidlaw in U.K. in 1933. The name “Influenza” originates from the Italian name for the disease: “influenza del freddo”, meaning “the influence of the cold”. The word influenza became English, in 1743 during an outbreak of this illness in Europe. Influenza is an old disease: Hippocrates clearly describes this disease some 2400 years ago. Like the common cold, influenza is highly contagious, both by droplets and contact with the sick person. Influenza is well known to cause both epidemics and pandemics, Three Influenza pandemics has occurred so far in the 20th century, each with new strains of the viruses, killing tens of millions of people.

Symptoms are usually acute chills, followed by high fever, sore throat, severe headache and muscle pains, cough, malaise and lethargy. In some cases gastro-intestinal symptoms are prominent, like nausea, vomiting and diarrhea, this type is often called the “stomach flu”. In the children, elderly and immune compromised persons (HIV infected persons, patients on chemotherapy for cancer), influenza could cause acute viral pneumonia which very often could be fatal. Flu is often loosely compared to common cold, but these are two different diseases, caused by different viruses, different clinical course and outcome (prognosis).

The economic impact of influenza is as impressive, or even more, than the common cold. In the U.S. an estimated 25-50 million flu cases are known to occur, which results in 150,000 hospitalizations and 30,000 to 40,000 mortality each year. Influenza costs the U.S annually $10 billion, including treatment, preventive measures, and lost productivity. The story would be very different, if a pandemic occurs: as exemplified by the 1918 Spanish Flu pandemic. It would be possible for 30% of the U.S. work force to get ill, they would be absent from work up to three weeks, and 2.5% of them could die from the illness. The gross domestic productivity in the U.S. would drop by 5% and adding the cost for treating the 18 to 45 million people who could get the infection, the total cost to the U.S. economy would add up to $700 billion.

In the treatment aspect, like common cold, flu has no specific treatment. All the symptomatic treatment used in cold works for flu as well. Antibiotics are not effective for influenza, but is used to treat secondary infections like pneumonia. Vaccinations are the only effective, preventive, treatment. Recently anti-viral drugs are being looked upon as some what effective. They are (A) Neuraminidase inhibitors: Oseltamivir(Tamiflu) and Zanamivir(Relenza). They are more useful in prevention, but do help to reduce the intensity of an attack and prevent some complications. The second antiviral drug used is (B) M2 inhibitors (Adamantanes), commercially called Amantidine and Rimantadine, their usefulness is limited, particularly because the viruses develop resistance to these drugs quickly (91% in 2005).

2. Description of the Related Art

The medical, herbal, and biochemical literature abound with text related to the use of the ingredients which formulate the total of this invention. The usefulness and dosages are provided with suggestions for a multitude of variations in formulations. It is within medical wisdom that more is definitely not necessarily better. This is applied to quantity of each component as well as to the quantity of separate components making up the whole. US patents issued on this subject range from utilizing only one ingredient to an extensive list of ingredients which include almost every type of herb, vitamin, and mineral. A well thought out, experimentally tested and proven formulation will provide more and better relief from the symptoms of the cold and flu than a remedy where everything goes. Still, it is important to learn from those already in practice and have received issued patents for their trials. It is for this reason that the following issued US patents are examined.

One of the ingredients within this invention will be found in other US patents:

Black pepper is used by Wolf for the deactivation of viruses and this is found in patent U.S. Pat. No. 4,592,910.

Cinnamon is found in several patents but not in combination with the ingredients used in this invention. Neeman teaches us that cinnamon can be used to inhibit the bacteria found associated with stomach ulcers and is noted in his patent U.S. Pat. No. 5,560,912. Levin includes cinnamon oil (patent U.S. Pat. No. 6,027,716) in an oral medication for the reduction of bacterial infections. Menon, in his patents U.S. Pat. No. 7,384,654 and U.S. Pat. No. 7,384,656, teaches the use of cinnamon for use in combating allergic reactions that produce flu like symptoms.

Ginger is found as a useful ingredient in many US patents for combating cold and flu like symptoms and associated symptoms of such. Emanuel-King teaches us the use of ginger along with other ingredients not found in this invention for the use of a tonic for use in fighting mouth bacteria and is found in patent U.S. Pat. No. 5,248,503. Kelly provides a teaching for the use of ginger as an ingredient in an oral tonic for the relief of pain from headaches and is found in patent U.S. Pat. No. 6,312,736. Boc utilizes ginger as an ingredient in a tonic for improving the health of an individual and this is found in patent U.S. Pat. No. 6,465,019. Fasano provides a method for better health through the ingestion of a tablet containing ginger and is found in patent U.S. Pat. No. 6,586,018. Watson, in patent U.S. Pat. No. 6,613,362, teaches the use of ginger in an herbal mix and used as a bowel soother during illness. Zhang proposed the use of ginger in a pharmaceutical preparation against type I allergies and is found in patent U.S. Pat. No. 6,641,846. Marchioni teaches the use of garlic in an oral preparation for improved respiration and is found in patent U.S. Pat. No. 7,087,250. Sha uses ginger in an oral preparation for fighting the flu and is found in patent U.S. Pat. No. 7,205,010.

Turmeric is used as an element in a health product is shown by several inventors. Schmitz utilizes turmeric in an edible form for health reasons as shown in patent U.S. Pat. No. 5,643,623. Nguyen does the same in his patent U.S. Pat. No. 6,048,533. Hastings utilizes turmeric for an oral compound to combat unhealthy joint functions as taught in patent U.S. Pat. No. 6,224,871. Yamamato has produced a tonic for providing a health drink using turmeric and teaches this in patent U.S. Pat. No. 6,416,807. Farley has produced a tonic that is suppose to provide natural immune functions using turmeric and this is taught in patents U.S. Pat. No. 6,544,564 and U.S. Pat. No. 6,767,563. Myhill teaches us the use of turmeric in an oral compound for the purpose of inflammation reduction and this is found in patents U.S. Pat. No. 7,241,461 and U.S. Pat. No. 7,384,655.

Rose hips is another ingredient of the present invention to be used as an anti-inflammatory and Vitamin C supply. Kharazmi teaches this as the primary ingredient in the patent U.S. Pat. No. 6,024,960. Although other components are added within the patent of Kharazmi, no other components associated with the present invention are found therein.

Two of the ingredients found within the formulary of this invention will be found together in the following US patents:

Black pepper and cinnamon. We are taught that the combination of these two ingredients de-activates cold and flu viruses. The teachings are found in the following patents of Wolf: U.S. Pat. No. 4,409,245 and U.S. Pat. No. 4,595,593. We are further taught that an energy supplement can be created using the two ingredients in a daily oral tonic by Keefe in patent U.S. Pat. No. 7,371,389.

Black pepper and turmeric. Majeed teaches us that the two ingredients increases the bioavailability of nutrient components and this is found in the three Majeed patents U.S. Pat. No. 5,536,506, U.S. Pat. No. 5,744,161, and U.S. Pat. No. 5,972,382.

Ginger and Turmeric. Without the utilization of the other components found in this invention, the use of these two are found in several patents. Patwardhan shows us a method using the two for muscle-skeletal discomfort in patent U.S. Pat. No. 5,494,668. Newmark utilizes the two as an inflammation reducer in the patents U.S. Pat. No. 6,264,995, U.S. Pat. No. 6,387,416 and U.S. Pat. No. 6,391,346. Krumhar also provides the same application for pain reduction and as a treatment for inflammation in patent U.S. Pat. No. 6,949,260. Rosenbloom applies the two for a nutritional supplement, an edible sore throat lozenge, and a method for the reduction of the transmission of an illness in patents U.S. Pat. No. 6,592,896, U.S. Pat. No. 6,596,313, U.S. Pat. No. 6,827,945 and U.S. Pat. No. 7,166,435. Rosenbloom further applies the two ingredients in a formulation as an anti-microbial in patent U.S. Pat. No. 7,396,546 and as a treatment for the flu virus in patent U.S. Pat. No. 7,405,046.

Three ingredients of the present invention are also found in combination within other US patents.

Black pepper, cinnamon, and turmeric. Wolf utilizes this combination in the preparation for the deactivation of viruses in patent U.S. Pat. No. 4,402,950.

Black pepper, cinnamon, and ginger. Dong uses this combination for use as an energy drink in patent U.S. Pat. No. 6,713,115.

Cinnamon, cumin, and ginger are used by Surburg in a compound to create a pleasant taste for such articles as toothpaste and is taught in patent U.S. Pat. No. 7,414,079.

Ginger, turmeric, and rose hips are used in combination by Foreman in a formulation for nervous disorders in patent U.S. Pat. No. 6,399,114.

We finally find formulations which use five of the ingredients of the present invention.

Black pepper, cinnamon, cumin, ginger, and rose hips. These are taught by Benet for use to increase the bioavailability of other pharmaceutical compounds. Such is found in patents U.S. Pat. No. 5,716,928 and U.S. Pat. No. 6,121,234.

The present invention utilizes seven active ingredients and an additional flavoring food to create an eight ingredient formulation. The ingredients are in many aspects, unique, in that many carry toxins which are removed before utilized so that toxic reactions are carefully forestalled. Carefully balanced, this new invention will provide relief to those who suffer from the symptoms of the common cold and flu.

### BRIEF SUMMARY OF THE INVENTION

Preparation of a novel herbal mixture for the symptomatic relief of common cold and flu is outlined in this preliminary patent application. Common cold and flu are very common illnesses and millions of people suffer from these illnesses every year. Professional and specialty organizations recommends against using commercially available drinks like colas, coffee, tea and alcoholic beverages. Keeping hydrated and keeping the airways clear and the temperature down are important steps to enhance recovery. There is no definitive treatment, at the present time, to both these illnesses.

### Components of the Cold and Flu Tonic

A summary of the separate ingredients to be used in this cold and flue tonic invention is provided for the purpose of understanding the usefulness of each, as well as defining the specific methods of extracting the necessary components from the structural whole of each. Although the components may seem simple, the precise biochemical extraction is part of a complex overall composition which must be precisely formulated for medical use.

Black Pepper (Piper nigrum) is a seed or fruit with seed spice.

Black Pepper is centuries old, its trade started 4000 years ago. Black pepper is grown almost exclusively in southern India, particularly Kerala State (old Malabar) and Indonesia. Its name, pepper, probably originated from the Sanskrit word “pippeli”. It has been later grown in some states in Africa. In ancient times tributes were paid in pepper, instead of cash, both Attila the Hun and Alaric I the Visigoth demanded pepper as a substantial part of Rome's ransom.

Black pepper carries the Latin name of Piper nigrum. It is from the plant kingdom known as Plantae, of the division Magnoliophyta, of the class Magnoliopsida, of the order Piperales of the family Piperaceae of the genus Piper, of the species known as P. nigrum. It carries the binomial name of Piper nigrum L.

The Piper nigrum plant is distributed throughout the tropical and subtropical forest regions of the world. The habitat of the naturally growing plant is the damp jungles on the slopes of Western Ghats in the Malabar Coast (India). About 30 species are known in India, of which only a few, including P. nigrum L (pepper), P. longum L (long pepper), P. cubeba L (cubeb), and P. betle L (pan, the leaves used for chewing), are known to be cultivated.

The Piper nigrum plant is a perennial woody vine growing to four meters in height on supporting trees, poles, or trellises. It is a spreading vine, rooting readily where trailing stems touch the ground. The leaves are alternate, dark green, ovate, acuminate, and thickish leaves approximately five to ten centimeters long and three to six centimeters broad. The flowers are small, produced on pendulous spikes four to eight centimeters long at the leaf nodes, the spikes lengthening to seven to 15 centimeters as the fruit matures. It is grown in soil that is neither too dry nor susceptible to flooding, moist, well-drained and rich in organic matter.

The Piper nigrum plants are propagated by cuttings about 40 to 50 centimeters long, tied up to neighboring trees or climbing frames at distances of about two meters apart. Trees with rough bark are favored over those with smooth bark, as the pepper plants with rough bark climb more readily. Competing plants are cleared away, leaving only sufficient trees to provide shade and permit free ventilation. The roots are covered in leaf mulch and manure, and the shoots are trimmed twice a year.

On dry soils the young plants require watering every other day during the dry season for the first three years. The plants bear fruit from the fourth or fifth year, and typically continue to bear fruit for seven years. The cuttings are usually cultivars, selected both for yield and quality of fruit. A single stem will bear 20 to 30 fruiting spikes.

The harvest begins as soon as one or two berries at the base of the spikes begin to turn red, and before the fruit is mature, but when full grown and still hard; if allowed to ripen, the berries lose pungency, and ultimately fall off and are lost. The spikes are collected and spread out to dry in the sun, then the peppercorns are stripped off the spikes.

The Piper nigrum produces stone fruits; green when unripe, then red and finally yellowish.

The vines reach heights of 4.5 to 7.5 m, climbing on supports (tree trunks or artificial props) through adventitious roots developing at swollen joints. Lateral hanging branches eventually form a dense cylindrical canopy of foliage. The inflorescence is borne on spikes of lengths varying from 5 to 20 cm, terminally on secondary and tertiary branchlets. Flowers are minute and are dioecious in wild and a few cultivated varieties or wholly perfect in many cultivated varieties. Fruits botanically called drupes, but generally called berries are ovoid or globose, dark green turning bright orange and red when ripe. The berry-like fruits, known a peppercorns, have a size about 0.5-1.0 cm in diameter and are developed on short, hanging spikes 4-12 cm. long. The varieties under cultivation have evolved by unconscious selection and show considerable variation in habitat, size and shape of fruit and fruiting behavior. Different harvest times and processing methods yield 4 types of pepper: black, green, white and red.

Black pepper is produced from the still-green almost not quite ripe berries of the pepper plant. The berries are cooked briefly in hot water, both to clean them and to prepare them for drying. The heat ruptures cell walls in the fruit, speeding the work of browning enzymes during drying. The berries are dried in the sun or by machine for several days, during which the fruit around the seed shrinks and darkens into a thin, wrinkled black layer, the result of a fungal reaction. Once dried, the fruits are called black peppercorns.

White pepper comes from the seed of the pepper berry. It is the same fruit, but harvested ripe and dried only after the hull is removed with the aid of soaking. The hull contains part of the volatile aroma compounds, while the pungency is located in the kernel only. White pepper therefore retains the full pungency of black pepper—or more, because of its ripeness—but it has an altered, less aromatic flavor. Because white pepper consists of the seed only, with the fruit removed, the fully ripe berry must then soak in water for about a week, during which the flesh of the fruit softens and decomposes. Rubbing then removes what remains of the fruit, and the naked seed is dried.

Green pepper is made from unripe green berries that are harvested much earlier than those harvested for making black pepper. The early harvested berries decay quickly. To retain their green color and freshness they must be quickly preserved in some fashion. The choice of preservation which also retains the green color consists of either pickling in brine or vinegar, treating with sulphur dioxide, or freeze drying. Because of its unripeness, green pepper has only light pungency, but it has a fresh, herbal, “green”, very aromatic flavor. The green piper nigrum berry is not to be confused with green capsicum or bell pepper which is also called “green pepper”; as it is an unrelated plant.

Red pepper is rare and created by the brine or vinegar pickling of the whole ripe red peppercorns (not to be confused with unrelated pink pepper), which combines the spicy, mature flavor of black pepper with the freshness of green pepper.

The main flavor of the peppercorn is from piperine, but other essential oils, including such terpenes as pinene, sabinene, limonene, caryophyllene, and linalool, also contribute to the aroma. The most powerful aromatic producing terpene is Rotundone, a bicyclic sesqui-terpene. Alkaloids found within the peppercorn include the pungent tasting chavicine and piperidine.

Medical use of pepper are numerous: carminative, stomach stimulant, anti-bacterial, diaphoretic, anti-flatulence, anti-nausea and taken during a chill (before fever) it helps raise the body temperature, averting a fever.

Piperine has been reported to inhibit drug-metabolizing enzymes, increases plasma concentrations and delays elimination of several drugs, including phenytoin and rifampin. By inhibiting drug metabolism, piperine may increase the bioavailability of various compounds. Particularly, piperine has been noted to enhance bioavailability of curcumin dramatically in humans.

Black Pepper, piper nigrum, is an FDA approved spice and is used in this invention for its anti-nausea, anti-bacterial, diaphoretic properties and as a drug metabolism delaying agent.

Cinnamon (Cinnamomum zeylanicum, aka: C. verum) is a tree bark spice.

Cinnamon comes from the bark of the Ceylon cinnamon tree, Cinnamomum zeylanicum, which is a small evergreen tree 10-15 meters (32.8-49.2 feet) tall, belonging to the kingdom Plantae, of the division Magnoliophyta, of the plant class Magnoliopsida, of the order Laurales, of the plant family Lauraceae, of the genus Cinnamomum, of the species C. verum with the binomial name Cinnamomum verum., is native to Sri Lanka and South India. The tree is also grown commercially at Tellicherry in southern India, Java, Sumatra, the West Indies, Brazil, Vietnam, Madagascar, Zanzibar, and Egypt.

The bark is widely used as a spice due to its distinct odor. In India it is also known as “Daalchini”. It is known as Canela in Portugal and Spain, Cannelle in France, and Zimt in Germany. In India and Iran, it is called Darchini, meaning “wood from China”, which more accurately describes Cassia, an alternate variety. The original name came from the Malay word “Kayumanis” meaning sweet wood. The Hebrew equivalent was “Qinnämön”, and this is the root of the word cinnamon. The word Canella was used by the Italians to describe “little cannon tubes” that the rolled up quills of bark resembled.

The name cinnamon is correctly used to refer to Ceylon Cinnamon, also known as “true cinnamon” (from the botanical name Cinnamomum zeylanicum). However, the related species Cassia (Cinnamomum aromaticum), Saigon Cinnamon (Cinnamomum loureiroi) and Cinnamomum burmannii are sometimes sold labeled as cinnamon, sometimes distinguished from true cinnamon as “Chinese Cinnamon”, “Vietnamese cinnamon” or “Indonesian cinnamon.” Ceylon cinnamon, using only the thin smooth inner bark, has a finer, less dense, and more crumbly texture with a light-yellowish-brown color, and is considered to be less strong than cassia. Cassia has a much stronger (somewhat harsher) flavor than Cinnamon and is generally a medium to light reddish brown, is hard and woody, in texture, and is thicker (2-3 mm thick), as all of the layers of bark are used.

The Egyptians used cinnamon and cassia along with myrrh in embalming, perhaps because cinnamic acid (and also myrrh) has antibacterial effects. The Hebrews, and others, used cinnamon and cassia in religious ceremonies, while in Mexico, Asiatic countries, Arabia and North Africa it was valued in cooking. The Roman empire imported huge amounts of cinnamon, and it may have been used mostly in perfumes and fragrances and to flavor wines, but it was not favored as a cooking spice.

The leaves are ovate-oblong in shape, 7-18 cm (2.7-7.1 inches) long. The flowers, which are arranged in panicles, have a greenish color, and have a distinct odor. The fruit is a purple one-centimeter berry containing a single seed.

Its flavor is due to an aromatic essential oil which makes up 0.5% to 1% of its composition. This oil is prepared by roughly pounding the bark, macerating it in seawater, and then quickly distilling the whole. It is of a golden-yellow color, with the characteristic odor of cinnamon and a very hot aromatic taste.

The pungent taste and scent come from cinnamic aldehyde or cinnamaldehyde and, by the absorption of oxygen as it ages, it darkens in color and develops resinous compounds.

Chemical components of the essential oil include ethyl cinnamate, eugenol, cinnamaldehyde, beta-caryophyllene, linalool and methyl chavicol.

The name cinnamon comes from Greek kinnammon, from Phoenician and akin to Hebrew qinnâmôn, itself ultimately from a Malaysian language, cf. Malay and Indonesian kayu manis which means sweet wood.

Cinnamon has been known from remote antiquity, and it was so highly prized among ancient nations that it was regarded as a gift fit for monarchs and other great potentates. It was imported to Egypt from China as early as 2000 BC, and is mentioned in the Bible in Exodus 30:23, where Moses is commanded to use both sweet cinnamon (Hebrew qinnmôn) and cassia, and in Proverbs 7:17-18, where the lover's bed is perfumed with myrrh, aloe and cinnamon, then lastly in Song of Solomon 4:14, a song describing the beauty of his beloved, cinnamon scents her garments like the smell of Lebanon. It is also alluded to by Herodotus and other classical writers. It was commonly used on funeral pyres in Rome, and the Emperor Nero is said to have burned a year's supply of cinnamon at the funeral for his wife Poppaea Sabina, in 65 AD.

Indonesian rafts transported cinnamon on a “cinnamon route” directly from the Moluccas to East Africa, where local traders then carried it north to the Roman market.

In the Middle Ages, the source of cinnamon was a mystery to the Western world. Arab traders brought the spice via overland trade routes to Alexandria in Egypt, where it was bought by Venetian traders from Italy who held a monopoly on the spice trade in Europe. The disruption of this trade by the rise of other Mediterranean powers, such as the Mamluk Sultans and the Ottoman Empire, was one of many factors that led Europeans to search more widely for other routes to Asia.

Portuguese traders finally discovered Ceylon (Sri Lanka) at the end of the fifteenth century, and restructured the traditional production of cinnamon by the salagama caste. The Portuguese established a fort on the island in 1518, and protected their own monopoly for over a hundred years.

Dutch traders dislodged the Portuguese by allying with the inland Ceylon kingdom of Kandy. They established a trading post in 1638, took control of the factories by 1640, and expelled all remaining Portuguese by 1658. “The shores of the island are full of it”, a Dutch captain reported, “and it is the best in all the Orient: when one is downwind of the island, one can still smell cinnamon eight leagues out to sea”. The Dutch East India Company continued to overhaul the methods of harvesting in the wild, and eventually began to cultivate its own trees.

The British took control of the island from the Dutch in 1796. However, the importance of the monopoly of Ceylon was already declining, as cultivation of the cinnamon tree spread to other areas, the more common cassia bark became more acceptable to consumers, and coffee, tea, sugar and chocolate began to outstrip the popularity of traditional spices. According to FAO, Indonesia produced almost 40% of the world cinnamon (canella) output in 2005 followed by China, India and Vietnam.

Cinnamon is harvested by growing the tree for two years and then coppicing it. The next year about a dozen shoots will form from the roots. These shoots are then stripped of their bark, which is left to dry. Only the thin (0.5 mm) inner bark is used; the outer woody portion is removed, leaving meter-long cinnamon strips that curl into rolls (“quills”) on drying; each dried quill comprises strips from numerous shoots packed together. These quills are then cut into 5-10 cm lengths for sale.

All of the powdered cinnamon sold in supermarkets in the United States is actually Cassia. European health agencies have recently warned against consuming high amounts of cassia, due to a toxic component called coumarin. Coumarin is known to cause liver and kidney damage in high concentrations. True Ceylon cinnamon, Cinnamomum zeylanicum, has negligible amounts of coumarin and thus the only cinnamon allowed to be used in this invention formulation.

The two barks, when whole, are easily distinguished, and their microscopic characteristics are also quite distinct. Cinnamon sticks (or quills) have many thin layers and can easily be made into powder using a coffee or spice grinder whereas cassia sticks are much harder, made up of one thick layer, capable of damaging a spice or coffee grinder. It is a bit harder to tell powdered cinnamon from powdered cassia. When powdered bark is treated with tincture of iodine (a test for starch), little effect is visible in the case of pure cinnamon of good quality, but when cassia is present a deep-blue tint is produced, the intensity of the coloration depending on the proportion of cassia.

In an evaluation of the antibacterial activity of cinnamon against six bacterial species of which included Escherichia coli, Klebsiella pneumoniae, Pseudomonas aeruginosa, Proteus vulgaris, Bacillus subtilis, and Staphylococcus aureus, cinnamon was found to inhibit all of them, even at low concentrations, and was determined to be a good antibacterial agent. In addition, a other studies of the antibacterial activity of cinnamon not only confirmed the action against the six bacterial species, but found cinnamon's effectiveness in inhibiting the growth of other bacteria including: Enterobacter aerogenes, Vibrio cholerae, Vibrio parahaemolyticus and Samonella typhymurium Cinnamon was also found effective against the fungi yeasts Candida albicans, Candida. tropicalis, Candida. glabrata, and Candida. krusei.

Coumarin free cinnamon, Cinnamomum zeylanicum, is an FDA approved spice and is utilized in this invention as an antibacterial ingredient.

Cumin (Cuminum cyminum) is a seed spice.

Cumin is the dried seed of the annual herbaceous plant Cuminum cyminum. The plant has a small and slender branched stem and is a member of the parsley family. It is from a flowering plant of the kingdom Plantae, of the plant division Magnoliophyta, the class of Magnoliopsida, of the order Apiales, in the family Apiaceae, the genus Cuminum, of the species C. cyminum and a native from the east Mediterranean to East India. It has the binomial name of Cuminum cyminum L.

The cumin plant grows to 30-50 cm (1-2 ft) tall and is harvested by hand. It is an herbaceous annual plant, with a slender branched stem 20-30 cm tall. The deep green leaves are 5-10 cm long, pinnate or bipinnate, thread-like leaflets resembling another culinary herb the fennel in form, each individual leaf is usually turned back at the tips. Leaf stalks tend to be absent from the upper leaves, but long leaf stalks are characteristic of the lower leaves.

Cumin bears small and white or rose-colored flowers, which have four to six rays on a stalked umbel, each individual flower being about half an inch long. The flowers are small, white or pink, and borne in umbels. The fruit is a laterall fusiform or ovoid achene 4-5 mm long, containing a single seed. Cumin seeds are similar to other herbal seeds. They are smaller and darker in color than fennel seeds, lighter in color than and not as smooth as caraway seeds. The shape of the seeds is oblong with a thicker middle part and laterally compressed tips; each individual seed is about one fifth of an inch in length. Cumin seeds are characterized by a peculiar odor and taste, hotter than that of caraway seeds.

Cultivation of cumin requires a long, hot summer of 34 months, with daytime temperatures around 30° C. (86° F.); it is drought tolerant, and is mostly grown in Mediterranean climates. It is grown from seed sown in spring, and needs a fertile, well-drained soil.

Cumin seeds are used as a spice for their distinctive aroma, popular in North African, Middle Eastern, Western Chinese, Indian, Cuban and Mexican cuisine. It is used as an ingredient of curry powder. Cumin can be found in some Dutch cheeses like Leyden cheese, and in some traditional breads from France. It is also wide-spread used by traditional culinary in Brazil. Cumin was also used heavily in ancient Roman cuisine.

Originally cultivated in Iran and Mediterranean region, cumin is mentioned in the Bible in both the Old Testament (Isaiah 28:27) and the New Testament (Matthew 23:23). It was also known in ancient Greece and Rome. The Greeks kept cumin at the dining table in its own container (much as pepper is frequently kept today), and this practice continues in Morocco. Cumin fell out of favor in Europe except in Spain and Malta during the Middle Ages. It was introduced to the Americas by Spanish colonists.

Cumin's distinctive flavor and aroma is due to its essential oil content with its main constituent compound: cuminaldehyde (4-isopropylbenzaldehyde).

Important aroma compounds of cumin are released after heating. These are the pyrazines:
2-ethoxy-3-isopropylpyrazine,
2-methoxy-3-sec-butylpyrazine, and
2-methoxy-3-methylpyrazine.

The cumin has effective and very strong stimulant properties; it is also a potent anti-spasmodic herb, as well as having carminative effects. The cumin was used formerly as an herbal remedy for treating colic and dyspeptic headaches.

A volatile oil present in the cumin is responsible for the strong aromatic odor and the warm, bitter taste of the cumin fruits, this oil is separated out from the cumin fruits by the process of distillation with water, the volatile oil makes up about two to four per cent of the cumin seeds. The volatile oil is formed by a chemical mixture of the compounds known as cymol or cymene as well as cuminic aldehyde, or cyminol (the chief constituent); the oil is limpid and pale yellow in coloration.

The cumin bears a fatty type of oil with resin, and substances such as mucilage and gum, as well as malates and albuminous matter in the tissues of the fruit, while a lot of the organic compound called tannin is contained in the outer seed coating of the cumin. Cumin gives an ash yield of about eight percent of dry weight.

Cumin, Cuminum cyminum, is an FDA approved spice and is used in this invention for its carminative, digestion stimulating, anti-oxidant, anti-spasmodic and sedative properties.

Ginger (Zingiber officinale) is a root spice.

Ginger is made from the “root” (rhizome) of the plant Zingiber Officinale, a monocotyledonous perennial plant. Ginger is a Latin word, derived from the Tamil word inji and the Malayalam word inchi (Tamil and Malayalam are south Indian languages). Ginger is from the plant kingdom Plantae, of the division Magnoliophyta, of the class Liliopsida, of the perennial plant order Zingiberales, from the family Zingiberaceae, of the genus Zingiber of the species Zinginer officinale.

Ginger plant originated in China and then went to India, south east Asia and West Africa. Today ginger is used world wide, including the U.S. pharmacies sell powered ginger, and every supermarket carries fresh ginger.

The culinary use of ginger is world wide, in every country ginger is used to cook a variety of food, as an additive to a variety of drinks, ginger ale is a common drink in the West, including the U.S.

Ginger is used for treating diseases from time immemorial. It is considered to be a carminative and stimulant (relieving gas and promoting digestion). It is known to reduce inflammation and arthritis, and reduce cholesterol and to some extent prevent blood clotting. FDA considers ginger as generally safe, but people taking Warfarin (blood thinner) should get medical guidance in the use of ginger. It relieves nausea and controls diarrhea. In the south of India, particularly in the state of Kerala, boiling dry ginger in water (chukku vellam) is used instead of tea or coffee for constantly sipping in a variety of febrile illnesses, obviously to prevent dehydration. It is served in many restaurants, instead of plain water, and the people of Kerala use chukku vellam (ginger water) just as the Japanese use green tea.

Fumonisins are toxins produced mainly by the molds Fusarium moniliforme (F. verticillioides), F. proliferatum, and several other Fusarium species that grow on agricultural commodities in the field or during storage. More than ten types of fumonisins have been isolated and characterized. Fumonisin B1 (FB₁), B2 (FB₂), and B3 (FB₃) are the major fumonisins produced. FB₁ is the most prevalent and most toxic. Recently and reported to the FDA in 2006, fumonisins have been found in botanical roots including ginger. Fumonisins have produced liver damage and changes in the levels of certain classes of lipids, especially sphingolipids, in all animals studied.

Many analytical methods for determining fumonisins in foods have been published. Among the most common of these methods are liquid chromatographic (LC) separation with fluorescence detection, enzyme-linked immunosorbent assay (ELISA) and LC/mass spectrometry. In our laboratory, the botanical roots of ginger were extracted with a mixture of methanol and water, followed by cleanup on an immunoaffinity column. FB₁ was then derivatized, separated and determined by LC with fluorescence detection. Recoveries of added FB₁ to turmeric at levels ranging from 0.25 to 2 ig/g were >85%. ELISA was also applied to screen these roots for FB₁.

This invention utilizes fumonisin-free ginger, Zingiber officinale, an FDA approved spice, as one of the ingredients for the treatment of cold and flu, due to its anti-inflammatory properties and in its control of nausea and diarrhea. It also provides relief to aches and pains and offers decongestion to the throat and nose.

Maple Syrup is a tree sap sugar food.

Maple syrup is derived from the sap of the maple trees of the kingdom Plantae, of the division Magnoliophyta, of the class Magnoliopsida, of the order Sapindales, of the family Aceraceae, of the genus Acer L. of the species Acer saccharum or Acer Nigrum.

Most of maple syrup is produced in Vermont in the United States and Quebec in Canada. The syrup contains about 67% solids, 89% of which is sucrose. The reminder is mainly fructose and glucose. It provides great nutritional value, sugar, calcium (more than milk) potassium (more than in bananas) magnesium, manganese, phosphorus, iron and thiamin (vitamin B₁), in addition to many amino acids (proteins) and phenolic compounds. A serving of 50 ml of maple syrup could provide the following percentage of daily allowances: calories 64, calcium 6%, Iron 5%, Manganese 2%, Thiamin 6% and Riboflavin 2%.

The caloric value of maple syrup is more than honey and corn syrup, volume for volume. Because of the high sugar content in maple syrup, it can also provide some effect as a preservative.

Maple Syrup, an FDA approved food, when applied in this invention is used as a sweetening agent and a preservative.

Rose hip, Rose Haw (Rosa canina) is a fruit Rose hip is the fruit of Rosa canina of the kingdom plantae of the plant division Magnoliophyta of the class Magnoliopsida of the plant order Rosales of the plant family Rosaceae of the genus Rosa L of the species Rosa canina.

Rose hip is a rich source of Vitamin C, about 1700-2000 mg per 100 gram Vitamin C has been considered to have clinical benefit with common cold and flu. Clinical studies however, has not conclusively proven this effect. But, use of Vitamin C in healthy adults who exercise regularly, is shown to have preventive effect for common cold. Earlier folks of the Americas have been using rose hips for treatment of colds and influenza because it has been shown to have anti-inflammatory effects.

In this invention, rose hip, Rosa canina, is an FDA approved fruit and is added as an ingredient, for providing natural vitamin C and as an anti-inflammatory. This is particularly useful for use long term, the inventor believes the cold and flu tonic is to be consumed daily, even after one recovers from cold and flu, to prevent or reduce the severity of further cold and flu attacks.

Saffron (Crocus sativus) a flower spice.

Saffron is a spice derived from the flower of the saffron crocus (Crocus sativus), of the plant kingdom Plantae, of the division Magnoliophyta, of the class Liliopsida, of the order Asparagales, of the family Iridaceae, of the genus Crocus, of the species Crocus sativus.

The flower has three stigmas, which are the distal ends of the plant's carpels. Together with its style, the stalk connecting the stigmas to the rest of the plant, these components are often dried and used in cooking as a seasoning and coloring agent. Saffron, which has for decades been the world's most expensive spice by weight, is native to Southwest Asia. It was first cultivated in the vicinity of Greece.

Saffron is characterised by a bitter taste and an iodoform- or hay-like fragrance; these are caused by the chemicals picrocrocin and safranal. It also contains a carotenoid dye, crocin, that gives food a rich golden-yellow hue. These traits make saffron a much-sought ingredient in many foods worldwide. Saffron also has medicinal applications.

The word saffron originated from the 12th-century Old French term safran, which derives from the Latin word safranum. Safranum is also related to the Italian zafferano and Spanish azafran.

The domesticated saffron crocus C. sativus is an autumn-flowering perennial plant unknown in the wild, and is a sterile triploid mutant of the eastern Mediterranean autumn-flowering Crocus cartwrightianus. According to botanical research, C. cartwrightianus originated in Crete. The saffron crocus resulted when C. cartwrightianus was subjected to extensive artificial selection by growers who desired elongated stigmas. Being sterile, the saffron crocus's purple flowers fail to produce viable seeds—thus, reproduction is dependent on human assistance: the corms (underground bulb-like starch-storing organs) must be manually dug up, broken apart, and replanted. A corm survives for only one season, reproducing via division into up to ten “cormlets” that eventually give rise to new plants. The corms are small brown globules up to 4.5 centimetres (1.8 in) in diameter and are shrouded in a dense mat of parallel fibers.

After a period of aestivation in summer, five to eleven narrow and nearly vertical green leaves—growing up to 40 cm (16 in) in length—emerge from the ground. In autumn, purple buds appear. Only in October, after most other flowering plants have released their seeds, does it develop its brilliantly hued flowers, ranging from a light pastel shade of lilac to a darker and more striated mauve. Upon flowering, it averages less than 30 cm (12 in) in height. Inside each flower is a three-pronged style; in turn, each prong terminates with a crimson stigma 25-30 mm in length.

The saffron crocus thrives in climates similar to that of the Mediterranean maquis or the North American chaparral, where hot, dry summer breezes blow across arid and semi-arid lands. Nevertheless, the plant can tolerate cold winters, surviving frosts as cold as 10 C (14 F) and short periods of snow cover. However, if not grown in wet environments like Kashmir (where rainfall averages 1000-1500 mm annually), irrigation is needed—this is true in the saffron-growing regions of Greece (500 mm of rainfall annually) and Spain (400 mm). Rainfall timing is also key: generous spring rains followed by relatively dry summers are optimal. In addition, rainfall occurring immediately prior to flowering also boosts saffron yields; nevertheless, rainy or cold weather occurring during flowering promotes disease, thereby reducing yields. Persistently damp and hot conditions also harm yields, as do the digging actions of rabbits, rats, and birds. Parasites such as nematodes, leaf rusts, and corm rot also pose significant threats.

Saffron plants grow best in strong and direct sunlight, but poorly in shady conditions. Thus, planting is best done in fields that slope towards the sunlight (i.e. south-sloping in the Northern Hemisphere), maximizing the crocuses' sun exposure. In the Northern Hemisphere, planting is mostly done in June, with corms planted some 7-15 cm deep. Planting depth and corm spacing—along with climate—are both critical factors impacting plant yields. Thus, mother corms planted more deeply yield higher-quality saffron, although they produce fewer flower buds and daughter corms. With such knowledge, Italian growers have found that planting corms 15 cm (5.9 in) deep and in rows spaced 2-3 cm apart optimizes threads yields, whereas planting depths of 8-10 cm optimizes flower and corm production. Meanwhile, Greek, Moroccan, and Spanish growers have devised different depths and spacings to suit their own climates.

After a period of dormancy through the summer, the corms send up their narrow leaves and begin to bud in early autumn. Only in mid-autumn do the plants begin to flower. Harvesting of flowers is by necessity a speedy affair: after their flowering at dawn, flowers quickly wilt as the day passes. Furthermore, saffron crocuses bloom within a narrow window spanning one or two weeks. Approximately one freshly picked flower yields 0.03 g of fresh saffron, or 0.007 g of dried saffron.

Saffron contains more than 150 volatile and aroma-yielding compounds. It also has many nonvolatile active components, many of which are carotenoids, including zeaxanthin, lycopene, and various carotenes. However, saffron's golden yellow-orange color is primarily the result of crocin. This crocin is trans-crocetin di-(-D-gentiobiosyl)ester (systematic (IUPAC) name: 8,8-diapo-8,8-carotenoic acid). This means that the crocin underlying saffron's aroma is a digentiobiose ester of the carotenoid crocetin. Crocins themselves are a series of hydrophilic carotenoids that are either monoglycosyl or diglycosyl polyene esters of crocetin. Meanwhile, crocetin is a conjugated polyene dicarboxylic acid that is hydrophobic, and thus oil-soluble. When crocetin is esterified with two water-soluble gentiobioses (which are sugars), a product results that is itself water-soluble. The resultant-crocin is a carotenoid pigment that may comprise more than 10% of dry saffron's mass. The bitter glucoside picrocrocin is responsible for saffron's flavor.

When saffron is dried after its harvest, the heat, combined with enzymatic action, splits picrocrocin to yield D-glucose and a free safranal molecule. Safranal, a volatile oil, gives saffron much of its distinctive aroma. Safranal is less bitter than picrocrocin and may comprise up to 70% of dry saffron's volatile fraction in some samples. A second element underlying saffron's aroma is 2-hydroxy4,4,6-trimethyl-2,5-cyclohexadien-1-one, the scent of which has been described as “saffron, dried hay like”. Chemists found this to be the most powerful contributor to saffron's fragrance despite its being present in a lesser quantity than safranal. Dry saffron is highly sensitive to fluctuating pH levels, and rapidly breaks down chemically in the presence of light and oxidizing agents. It must therefore be stored away in air-tight containers in order to minimize contact with atmospheric oxygen.

Saffron types are graded by quality according to laboratory measurements of such characteristics as crocin (color), picrocrocin (taste), and safranal (fragrance) content. Other metrics include floral waste content (i.e. the saffron spice sample's non-stigma floral content) and measurements of other extraneous matter such as inorganic material (“ash”).

A uniform set of international standards in saffron grading was established by the International Organization for Standardization, which is an international federation of national standards bodies. Namely, ISO 3632 deals exclusively with saffron. It establishes four empirical grades of color intensity: IV (poorest), III, II, and I (finest quality). Saffron samples are then assigned to one of these grades by gauging the spice's crocin content, which is revealed by measurements of crocin-specific spectroscopic absorbance.

Medicinally, saffron has a long history as part of traditional healing; modern medicine has also discovered saffron as having anticarcinogenic (cancer-suppressing), anti-mutagenic (mutation-preventing), immunomodulating, and antioxidant-like properties.

In this invention, the ingredient saffron, Crocus sativus, is an FDA approved spice and is used for its antioxidant-like properties and as an anticarcinogenic.

Turmeric (Curcuma longa) is a root spice.

Turmeric is from a herbaceous perennial plant of the ginger family and of the kingdom Plantae, of the division Magnoliophyta, of the class Liliopsida, of the order Zingiberales, of the family Zingiberaceae, of the genus Curcuma, of the species C. longa with the binomial name Curcuma longa.

The rhizome of the root is the part of the plant collected, boiled for several hours and then dried in hot ovens, followed by grinding it to a deep orange-yellow powder. It is an integral part of every cooking curry powder, and is widely used in India. Use of turmeric dates as far back as the Vedic era (4000 BC) in India. The state of Maharashtra in India, is the main trading center for turmeric. Its main active ingredient is curcumin.

In addition to its culinary applications, turmeric has come to the fore front in modern medicine as a potential treatment for infections, liver disorders, Alzheimer's disease, depression, psoriasis, and a variety of cancers: multiple myeloma, pancreatic cancer, breast cancer and colo-rectal cancer. It is being used in cosmetic powders and pastes. The degree of keen interest turmeric has raised in the modern medical community is reflected in the two hundred and fifty six papers published in 2006 alone, as published by the Wikipedia Encyclopedia, after a search with the U.S. National Library of Medicine.

Curcuminoids are polyphenolic pigments found in the spice turmeric. The term turmeric is used both for the plant Curcuma longa Linnaeus and the spice derived from the rhizomes of the plant.

Curcumin (diferuloylmethane) is an orange-yellow component of turmeric (Curcuma longa), a spice often found in curry powder. In recent years, considerable interest has been focused on curcumin due to its use to treat a wide variety of disorders without any side effects. It is one of the major curcuminoids of turmeric, which impart its characteristic yellow color. Curcumin has the potential to treat a wide variety of inflammatory diseases through modulation of numerous molecular targets.

Taken orally, turmeric inhibits the response of the body to inflammatory agents both directly and indirectly, by stimulating production of natural corticosteroids from the adrenal glands, “sensitising” cortisone receptor sites on cells, and preventing the breakdown of cortisol. In fact, curcumin has been found to be every bit as effective as the pharmaceutical drugs cortisone and phenylbutazone in clinical trials. Unlike anti-inflammatory pharmaceuticals, curcumin has no known side effects.

The major curcuminoids comprise 3-6% of Turmeric and of this percentage,
70-75% is Curcumin,
15-20% is Demethoxycurcumin
3% is Bisdemethoxycurcumin.

The Turmeric herb has been used in clinical trials. All the clinical trials point to one thing—1,200mg of turmeric extract is required before a human receives the needed blood levels to influence pain and inflammation positively. Therein lies a big problem as tumeric is a huge molecule that is nearly insoluble and thus poorly absorbed. In fact, it was shown that less than 1 per cent is absorbed. To receive the required blood levels, specialized techniques are required and available today.

The active constituent is curcumin. It has been shown to have a wide range of therapeutic effects. First, it protects against free radical damage because it is a strong antioxidant. It reduces inflammation. It accomplishes this by reducing histamine levels and by increasing production of natural cortisone by the adrenal glands. It protects the liver from a number of toxic compounds. It has been shown to reduce platelets from clumping together, which in turn, improves circulation and helps protect against atherosclerosis. Numerous studies have also shown cancer-preventing effects of curcumin. This may be due to its powerful antioxidant activity in the body. As a powerful antioxidant it has shown greater effects in preventing free radical damage compared to vitamin C, vitamin, E and superoxide dismutase.

Many studies on turmeric have revealed that the herb contains cholagogue-type substances which increase the secretion of bile. Principal among these substances is curcumin which also possesses liver protective activity, detoxifying dangerous carcinogens, stimulating the gall bladder and acting as a free-radical scavenger. Curcumin has cholekinotic activity (bile duct stimulation). It has been suggested that turmeric lowers blood cholesterol through these various choleric effects. Turmeric's effect on weight loss may also be mediated through curcumin's catabolic and metabolic activities on fats. Studies have also revealed that curcumin has anti-inflammatory properties, inhibiting platelet aggregation and cyclooxgenase and lipoxygenase enzymes which catalyze the formation of inflammatory prosteglandins and molecules. Curcumin requires the presence of the adrenal glands to have this non-steroidal anti-inflammatory activity.

Curcumin is thought to be a powerful antinociceptive (pain-relieving) agent. Studies have shown the effectiveness of turmeric in the reduction of joint inflammation, and recommended clinical trials as a possible treatment for the alleviation of arthritis symptoms. It is thought to work as a natural inhibitor of the cox-2 enzyme, and has been shown effective in animal models for neuropathic pain secondary to diabetes, among others.

Fumonisins are toxins produced mainly by the molds Fusarium moniliforme (F. verticillioides), F. proliferatum, and several other Fusarium species that grow on agricultural commodities in the field or during storage. More than ten types of fumonisins have been isolated and characterized. Fumonisin B₁ (FB₁), B2 (FB₂), and B3 (FB₃) are the major fumonisins produced. FB₁ is the most prevalent and most toxic. Recently and reported to the FDA in 2006, fumonisins have been found in botanical roots including turmeric. Fumonisins have produced liver damage and changes in the levels of certain classes of lipids, especially sphingolipids, in all animals studied.

Many analytical methods for determining fumonisins in foods have been published. Among the most common of these methods are liquid chromatographic (LC) separation with fluorescence detection, enzyme-linked immunosorbent assay (ELISA) and LC/mass spectrometry. In our laboratory, the botanical roots of turmeric were extracted with a mixture of methanol and water, followed by cleanup on an immunoaffinity column. FB₁ was then derivatized, separated and determined by LC with fluorescence detection.

For this invention, the application of fumonisin-free turmeric, Curcuma longa, is an FDA approved spice and is used for its anti-inflammatory, anti-bacterial, digestive and nutritional values.

### DETAILED DESCRIPTION OF THE INVENTION

This invention fills a vacuum in the requirement for symptomatic treatment of the common cold and flu and describes the combined use of several herbal items, ginger, pepper, cinnamon, cumin seeds, rose hips, black pepper and saffron with maple syrup as a potential sweetening agent. This invention calls this mixture “Cold and Flu Tonic”. The tonic is defined as either a compound in liquid form or a concentrate in dry powder form for re-constitution into a liquid form at a later date by the user.

The indications for this “Cold and Flu Tonic” is not only acute common cold and Influenza, for symptomatic relief and hydration, but this tonic is also designed to be a daily drink, after the acute attack, to prevent or reduce the severity of further attacks of cold and flu.

Example of one method of hot extraction into decoction, depending upon the formulation, is to sliced or mince ginger, turmeric, rose hip, ground cumin seeds and sliced fresh black peppercorn will be boiled in a filtered aqueous solution at 100 degrees centigrade for 5 to 15 minutes. Saffron and/or ground cinnamon bark would be added per formula. The jars are then securely covered and stored at room temperature for 24 to 48 hours. Maple syrup would be then added, in a proportion of 8:1 or 6:1, 4:1 or 3:1, as required to control the flavor and sweetness. The jars will be sealed again, irradiated for sterility and using a sterile pump the mixture would be transferred to bottles, capped and sealed. It is also possible to irradiate the mixture after it is bottled per FDA standards for seed and herb tolerances.

Another method would be the use of cold filtration and pressure extraction. In mass production this methodology would be modified using large stainless steel tanks, heat exchange or cold pressure and filtration and pumps to transfer the mixture, by “no touch” technology. The resultant component to be distributed in said liquid form, concentrated liquid form or a liquid-removed dry powder concentrate for later re-constitution into a useable liquid form.

I claim:

1. A composition for reducing the symptoms of the common cold and flu in a human subject in need of, comprising the administration of an effective amount of an aqueous composition having all components dispersed in a dry powder concentrate or dissolved in a decoction therein comprising Black pepper, Cumin, Ginger, Rose hip, and Turmeric.

2. A composition for reducing the symptoms of the common cold and flu in a human subject in need thereof, comprising the administration of an effective amount of an aqueous composition having all components dispersed in a dry powder concentrate or dissolved in a decoction therein comprising Black pepper, Cinnamon, Cumin, Ginger, Rose hip, and Turmeric.

3. A composition for reducing the symptoms of the common cold and flu in a human subject in need thereof, comprising the administration of an effective amount of an aqueous composition having all components dispersed in a dry powder concentrate or dissolved in a decoction therein comprising Black pepper, Cinnamon, Cumin, Ginger, Rose hip, Saffron, and Turmeric.

4. The composition of claim 1 wherein said Black pepper is the fruit and seed of Piper nigrum and is present in said decoction in the expected range of from 10 mg to 50 mg and a target dose of 20 mg per total final aqueous volume of 1 liter.

5. The composition of claim 1 wherein said Cumin is the seed containing cuminaldehyde of Cuminum cyminum and is present in said decoction in the expected range of from 25 mg to 100 mg and a target dose of 35 mg per total final aqueous volume of 1 liter.

6. The composition of claim 1 wherein said ginger is the rhizome of Zingiber officinale and contains no toxic fumonisins and is present in said decoction in the expected range of from 35 mg to 100 mg and a target dose of 75 mg per total final aqueous volume of 1 liter.

7. The composition of claim 1 wherein said Rose hip is the fruit of the Rosa canina and is present in said decoction in the expected range of from 15 mg to 75 mg and a target dose of 25 mg per total final aqueous volume of 1 liter.

8. The composition of claim 1 wherein said Turmeric is the rhizome of Curcuma longa and contains no toxic fumonisins and is present in said decoction in the expected range of from 10 mg to 50 mg and a target dose of 15 mg per total final aqueous volume of 1 liter.

9. The composition of claim 1 further comprising maple syrup in said decoction made from the sap of the maple trees of the species Acer saccharum or Acer Nigrum and is present in said decoction in the expected range of from 25 ml to 100 ml and a target dose of 40 ml per total final aqueous volume of 1 liter.

10. The composition of claim 2 wherein said Black pepper is the fruit and seed of Piper nigrum and is present in said decoction in the expected range of from 10 mg to 50 mg and a target dose of 20 mg per total final aqueous volume of 1 liter.

11. The composition of claim 2 wherein said Cumin is the seed containing cuminaldehyde of Cuminum cyminum and is present in said decoction in the expected range of from 25 mg to 100 mg and a target dose of 35 mg per total final aqueous volume of 1 liter.

12. The composition of claim 2 wherein said ginger is the rhizome of Zingiber officinale and contains no toxic fumonisins and is present in said decoction in the expected range of from 35 mg to 100 mg and a target dose of 75 mg per total final aqueous volume of 1 liter.

13. The composition of claim 2 wherein said Rose hip is the fruit of the Rosa canina and is present in said decoction in the expected range of from 15 mg to 75 mg and a target dose of 25 mg per total final aqueous volume of 1 liter.

14. The composition of claim 2 wherein said Turmeric is the rhizome of Curcuma longa and is present in said decoction in the expected range of from 10 mg to 50 mg and a target dose of 15 mg per total final aqueous volume of 1 liter.

15. The composition of claim 2 wherein said Cinnamon is formed of the inner bark of Cinnamomum zeylanicum, of the species C. verum with the binomial name Cinnamomum verum and contains no toxic Coumarin and is present in said decoction in the expected range of from 15 mg to 50 mg and a target dose of 25 mg per total final aqueous volume of 1 liter.

16. The composition of claim 2 further comprising maple syrup in said decoction made from the sap of the maple trees of the species Acer saccharum or Acer Nigrum and is present in said decoction in the expected range of from 25 ml to 100 ml and a target dose of 40 ml per total final aqueous volume of 1 liter.

17. The composition of claim 3 wherein said Black pepper is the fruit and seed of Piper nigrum and is present in said decoction in the expected range of from 10 mg to 50 mg and a target dose of 20 mg per total final aqueous volume of 1 liter.

18. The composition of claim 3 wherein said Cumin is the seed containing cuminaldehyde of Cuminum cyminum and is present in said decoction in the expected range of from 25 mg to 100 mg and a target dose of 35 mg per total final aqueous volume of 1 liter.

19. The composition of claim 3 wherein said ginger is the rhizome of Zingiber officinale and contains no toxic fumonisins and is present in said decoction in the expected range of from 35 mg to 100 mg and a target dose of 75 mg per total final aqueous volume of 1 liter.

20. The composition of claim 3 wherein said Rose hip is the fruit of the Rosa canina and is present in said decoction in the expected range of from 15 mg to 75 mg and a target dose of 25 mg per total final aqueous volume of 1 liter.

21. The composition of claim 3 wherein said Turmeric is the rhizome of Curcuma longa and contains no toxic fumonisins and is present in said decoction in the expected range of from 10 mg to 50 mg and a target dose of 15 mg per total final aqueous volume of 1 liter.

22. The composition of claim 3 wherein said Cinnamon is formed of the inner bark of Cinnamomum zeylanicum, of the species C. verum with the binomial name Cinnamomum verum and contains no toxic Coumarin and is present in said decoction in the expected range of from 15 mg to 50 mg and a target dose of 25 mg per total final aqueous volume of 1 liter.

23. The composition claim 3 wherein Saffron is the flower component of Crocus sativus and is present in said decoction in the expected range of from 10 mg to 50 mg and a target dose of 10 mg per total final aqueous volume of 1 liter.

24. The composition of claim 3 further comprising maple syrup in said decoction made from the sap of the maple trees of the species Acer saccharum or Acer Nigrum and is present in said decoction in the expected range of from 25 ml to 100 ml and a target dose of 40 ml per total final aqueous volume of 1 liter.
